(19) United States
(12) Patent Application Publication
(10) Pub. No.: US 2025/0043348 A1
(43) Pub. Date: Feb. 6, 2025

(54) AN EPIGENETIC CLOCK FOR THE GALLIFORMES FAMILY

(71) Applicant: Evonik Operations GmbH, Essen (DE)

(72) Inventors: Suki ROY, Singapore (SG); Sanjanaa NAGARAJAN, Singapore (SG); Frank LYKO, Heidelberg (DE); Florian BÖHL, Neckargemünd (DE); Günter RADDATZ, Heidelberg (DE); Sina LYKO-TÖNGES, Heidelberg (DE); Jennifer BOURLAND, Singapore (SG); Walter PFEFFERLE, Langgöns (DE); Rose WHELAN, Ambrosden (GB)

(73) Assignee: Evonik Operations GmbH, Essen (DE)

(21) Appl. No.: 18/836,232

(22) PCT Filed: Feb. 1, 2023
(86) PCT No.: PCT/EP2023/052394

§ 371 Date: Aug. 6, 2024

(30) Foreign Application Priority Data
Feb. 7, 2022 (EP) 22155373.8

(51) Int. Cl. C12Q 1/6881; C12Q 1/6806; C12Q 1/686

(52) U.S. Cl. CPC C12Q 1/6881; C12Q 1/6806; C12Q 1/686; C12Q 2600/154

(57) ABSTRACT

An in vitro method for predicting the biological age of a subject includes: (a) bisulfite treatment of genomic DNA extracted from a biological sample obtained from the subject; (b) amplifying the bisulfite treated genomic DNA from step (a) with specific primers that target at least two Low Methylated Region (LMR) in the genomic DNA from (a) to obtain at least one PCR product; (c) measuring the methylation level of CpG sites in the PCR product of step (b); and (d) determining the biological age of the subject with a statistical prediction algorithm, wherein the statistical prediction algorithm is applied to the measured methylation level to determine the epigenetic age of the subject; wherein the subject is from the Galliformes family; and wherein at least three LMRs are amplified in step (b); and with a proviso that the CpG sites associated with single nucleotide polymorphisms are not considered.

### FIELD OF THE INVENTION

The present invention relates to a method for establishing the epigenetic age of Galliformes. In particular, the method uses PCR in combination with a minimum number of Low Methylated Regions (LMRs) to form a DNA mini clock that is used to determine the biological age of a subject from the Galliformes family, for example chicken (Gallus gallus domesticus).

### BACKGROUND OF THE INVENTION

In the current socioeconomic global situation, there is strong evidence of public concern over the moral implications of actual animal production systems on farm animal welfare. As proof that the animal-ethics dimension of sustainability is considered, the United Nations Committee on World Food Security stated in its ‘Proposed draft recommendations on sustainable agricultural development for food security and nutrition including the role of livestock’, Recommendation ‘D’ of Article VIII, entitled ‘Animal health and welfare’: “Improve animal welfare delivering on the five freedoms and related OIE standards and principles, including through capacity building programs, and supporting voluntary actions in the livestock sector to improve animal welfare”.” (Alonso, M. E. et al. (2020) Animals, 10:385.). In 2002, the World Organization for Animal Health (OIE) expanded its mandate to become the leading international body in the field of animal welfare (Lopez J. (2007), Can Vet J., 48 (11): 1163-64.). Subsequently, animal welfare was illuminated from different angles including global issues, trends, and challenges.

“Currently, the OIE is the primary international standard-setting organisation for veterinary concerns and it provides guidelines, codes and science-based standards for various aspects of animal health to member states. The OIE ensures that animal welfare is an international priority through the OIE Terrestrial Animal Health Code (Terrestrial Code). The OIE defines animal welfare as “how an animal is coping with the conditions in which it lives. An animal is in a good state of welfare if (as indicated by scientific evidence) it is healthy, comfortable, well nourished, safe, able to express innate behavior, and if it is not suffering from unpleasant states such as pain, fear, and distress”.” (Cornish, A. (2016), Animals: 6 (7)).

A major challenge remains on how to measure or quantify animal welfare beyond standard operating procedures or guiding measures. Though these approaches aim to improve the environment of livestock animals, it is unclear whether welfare increases from an animal-centric perspective. The biological functioning and affective state frameworks were initially seen as competing, but a recent more unified approach is that biological functioning is taken to include affective experiences and affective experiences are recognized as products of biological functioning, and knowledge of the dynamic interactions between the two is considered to be fundamental to managing and improving animal welfare” (Hemsworth P H, et al. (2015), N Z Vet J.; 63 (1): 24-30).

It has been shown that the environment affects gene expression and phenotypes, both in plants and animals. Different environmental cues (such as nutrition, chemical compounds, temperature changes and other stresses) can affect phenotypes and epigenetic gene regulation in experimental model systems. The underlying mechanism is called epigenetics—the modification of genes via chemical marks on the DNA, post translational modification of histones or RNA molecules (miRNA). Epigenetic modifications are induced by the environment in the broad sense: the cell constantly receives all kinds of signals informing it about its environment, so that it specializes during development, or adjusts its activity to the situation. These signals can lead to changes in the expression of our genes, without affecting their genetic sequence and result in a so-called phenotype.

Furthermore, Steve Horvath (2013) underpinned the role of epigenetics in senescence as he found that DNA methylation correlates with chronological age (Horvath S. (2013) Genome Biol.; 14 (10): R115) which is the basis for “epigenetic clocks” that can estimate the DNA methylation age/epigenetic age in specific tissues or tissue-independently and can predict mortality and time left before death. Epigenetic age is highly correlated with the biological age and respond to environmental factors that accelerate or decelerate ageing processes, resulting in substantial deviations from the ‘real’ biological age. Hence, environmental confounders or cues contribute to either age acceleration or age deceleration whereas, age acceleration matches up with negative confounders/factors such as disease, stress, malnutrition, negative environmental conditions, etc. In contrast, both well-being and healthy nutrition tie in with age deceleration.

Epigenetic age acceleration (epigenetic age>chronological age) suggests that the underlying tissue ages faster than expected on the basis of chronological age, whereas a negative value (epigenetic age<chronological age, age deceleration) suggests that the tissue ages slower than would be expected. Epigenetic age acceleration is associated with a great number of age-related conditions and diseases, such as inflammatory processes.

Low-methylated regions (LMRs) represent a key feature of the dynamic methylome. LMRs are local reductions in the DNA methylation landscape and represent CpG-poor distal regulatory regions that often reflect the binding of transcription factors and other DNA-binding proteins. LMRs were originally described for the mouse (Stadler et al. (2011) Nature: 480, 490-95). Evolutionary conservation of LMRs beyond mammals has remained unexplored.

Galliformes, such as chicken (Gallus gallus domesticus), are a significant source of commercially produced meat and eggs. Accordingly, the welfare of the birds in this species is of great concern to the public. Raddatz et al. has developed as an example for animal livestock a multi-tissue predictor for age which works for chicken, too (Raddatz, G., et al., (2021) Commun Biol: 4 (76)). The author could demonstrate that a chicken methylation clock with 32 LMRs could predict at a high precision the health status of an animal. In particular, samples from inflamed tissues showed significant age acceleration.

Accordingly, there is a need in the art for a simple and reliable method based on epigenetics for measuring or quantifying animal welfare beyond standard operating procedures or guiding measures for Galliformes, such as chicken, turkey, quail or pheasants. In particular there is a need for means of measuring the welfare of a bird from the Galliformes family with improved specificity, accuracy and precision; and to provide a method for establishing the inflammation status, respectively.

### DESCRIPTION OF THE INVENTION

The present invention attempts to solve the problems above by providing a method that is capable to identify the epigenetic age of a subject from the Galliformes family. In particular, the method according to any aspect of the present invention amplifies specific Low Methylated Regions (LMR) from DNA of a biological sample of a subject from the Galliformes family using PCR and then determines the methylation level of the CpG sites in the PCR product to predict the age of the subject. Keeping a methylation clock (i.e. an LMR-based DNA methylation clock), in particular a ‘mini clock’ where a minimum number of LMRs are needed to predict the epigentic age, using the method according to any aspect of the present invention may thus be a suitable tool to measure animal welfare of the subject from the Galliformes family and to maintain good/excellent animal welfare during the course of life of the animal Galliformes family.

This LMR-based mini-clock (known as a mini clock since only a minimum number of LMRs are needed) is not only fast but also precise at predicting the biological age of the sample/subject. If animal keeping conditions lead to aging (as previously demonstrated by Raddatz et al. (2021)), this can be easily measured by this minimum sized LMR based methylation clock and correlated to the welfare status of an animal flock as average age relates to a normal welfare status while a significantly accelerated age corresponds to below average animal welfare. Finally, age deceleration matches up to excellent animal welfare.

In particular, the present invention relates to methods for estimating the biological age of an individual tissue or cell type sample from an animal of the Galliformes family based on measuring DNA Cytosine-phosphate-Guanine (CpG) methylation levels of LMRs in the DNA of the sample. In one example, a method is disclosed comprising a first step of choosing a biological cell or tissue sample from an animal of Galliformes family. In a second step, genomic DNA is extracted from the collected tissue of the animal from which an age prediction is desired. In a third step, the methylation levels of the CpG sites at the LMRs are measured. In a fourth step, a statistical prediction algorithm is applied to the methylation levels to predict the biological age. One basic approach is to form a weighted average of the CpGs, which is then transformed to DNAm age using a calibration function.

According to one aspect of the present invention, there is provided an in vitro method for predicting the biological age of a subject, the method comprising the steps of:
(a) bisulfite treatment of genomic DNA from a biological sample obtained from the subject;
(b) amplifying the bisulfite treated genomic DNA from step (a) with specific primers that target at least two Low Methylated Region (LMR) in the genomic DNA from (a) to obtain at least one PCR product;
(c) measuring the methylation level of CpG sites in the PCR product of step (b);
(d) comparing the measured methylation level of the CpG sites form step (c) with reference methylation levels of the CpG sites to determine the biological age of the subject based on the methylation levels,
wherein the subject is from the Galliformes family and
   with a proviso that the CpG sites associated with single nucleotide polymorphisms are not considered.

More in particular, according to one aspect of the present invention, there is provided an in vitro method for predicting the biological age of a subject, the method comprising the steps of:
(a) bisulfite treatment of genomic DNA extracted from a biological sample obtained from the subject;
(b) amplifying the bisulfite treated genomic DNA from step (a) with specific primers that target at least two Low Methylated Region (LMR) in the genomic DNA from (a) to obtain at least one PCR product for each LMR;
(c) measuring the methylation level of CpG sites in the PCR product of step (b);
(d) determining the biological age of the subject with a statistical prediction algorithm, wherein the statistical prediction algorithm is applied to the measured methylation level to determine the epigenetic age of the subject
   wherein the subject is from the Galliformes family and
   wherein the wherein at least three LMRs are amplified in step (b), and
   with a proviso that the CpG sites associated with single nucleotide polymorphisms are not considered.

The method according to any aspect of the present invention has the advantage that a minimum of only 3 LMRs (relative to 32 LMRs) are required to determine the biological age of the test animal. Accordingly, lesser processing time is required to generate the data and determine the biological age of the animal.

Further, in the past, especially in WO 2021/148601, for the 32 LMRs disclosed, the only option for data generation was WGBS, as even RRBS was shown not to have captured all 32 LMRs. WGBS is expensive, time consuming, computationally intensive and lacks coverage uniformity. Bisulfite PCR used according to any aspect of the present invention on the other hand, is cheaper, quicker, computationally less intensive and guarantees the respective coverage to build a precise clock.

In particular, to generate the data required to determine the biological age of the test animal with PCR, each sample requires multiple (approximately 3) PCR reactions for each LMR due to the large size of each LMR. For 32 LMRs as required in WO 2021/148601, this would require more than 90 reactions per sample to generate the data, while with only 3 LMRs as required by the method according to any aspect of the present invention, less than 10 PCR reactions per sample are required. 3 of the original 32 LMRs as shown by the examples has the efficiency of calculating the biological age with a minor but still acceptable standard deviation.

Use of only 3 LMRs also allows for data generation from cheaper, quicker sequencing technologies like Sanger sequencing. Bisulfite sanger sequencing creates much smaller sequence data sets than WGBS or RRBS, which can be quantified to methylation ratios and then used to calculate the biological age. Bisulfite sanger sequencing of a 3 LMR mini gallus clock would therefore offer another cheaper, quicker and more robust methodology in comparison to processing 32 LMRs with either bisulfite PCR, bisulfite Sanger or WGBS.

For array-based technologies the challenges in designing a 32 LMR gallus clock is that not all CpG sites are designable for probes. A reduction in total LMRs required for a clock would increase the probability that a gallus clock could be designed for an array as fewer CpG sites are required. Accordingly, the use of a mini clock in conjunction with orthogonal methods (bisulfite PCR or sanger sequencing) for data quantification provides the accessible, unique and reliable method for biological age determination.

In one example, the method according to any aspect of the present invention is summarized in FIG. 3.

The inventors have found that robustness, specificity, accuracy and precision of the LMR-based mini clock for Galliformes family can be significantly improved by (i) excluding from the initially identified clock CpG sites all CpG sites associated with single nucleotide polymorphisms (SNPs), (ii) excluding all CpG sites located on the sex chromosomes (Z and W), and (iii) normalizing the CpG methylation values.

Galliformes is an order of heavy-bodied ground-feeding birds which includes turkey, grouse, chicken, ptarmigan, quail, partridge, pheasant, francolin, junglefowl and the Cracidae. This order contains five families: Phasianidae (including chicken (Gallus gallus domesticus), quail, partridges, pheasants, turkeys, peafowl and grouse), Odontophoridae, Numidiae, Cracidae and Megapodiiae.

In particular, establishment of a chicken DNA methylation clock is carried out using a method disclosed in (Raddatz, G., Commun Biol 4, 76 (2021). Briefly, in order to establish a chicken methylation clock, a penalized regression model (implemented in the R-package glmnet (Friedman, J., et al., J. Stat. Softw. 33, 1-22 (2010)) is applied to regress the chronological age of animals tested on the normalized methylation values of the CpG probes. This approach, which computationally assigns weights to the set of CpG probes and thus selects an optimized set of markers, was established in the seminal paper by Horvath (Horvath, S. et al., Nat. Rev. Genet 19, 371-384 (2018)) and has since been applied in nearly all studies on DNA methylation clocks. In the case of the LMR-based DNA methylation clock, a penalized regression model was applied to regress the chronological age of the animals on the normalized average methylation values of the LMRs.

From the LMR-based DNA methylation clock, an LMR-based mini-clock was established according to any aspect of the present invention. This mini-clock is then trained to determine the biological age of the sample in question based on the size of the PCR products obtained from the biological sample. In particular, the biological age of the subject is computed by summation of the products of average methylation value and weight of the LMRs.

The term “chronological age” refers to the calendar time that has passed from birth/hatch.

The biological age depends on the biological state or condition of an individual or of a population and takes into account the circumstances of life (such as stress, nutrition, etc.). The terms “epigenetic age”, “methylation age”, and “biological age” have identical meanings and are used interchangeably in the context of the present application.

The method for predicting the chronological age of Galliformes can be used for testing individual subjects and for testing a complete animal population, such as a chicken or broiler/layer flock.

The biological sample material derived from the subject or from the population to be tested may be, for example, selected from the group consisting of body fluids, excremental material, tissue material, such as muscle tissue, gut tissue, organ tissue, skin tissue, feather material, such as quill pen, or combinations thereof. Excremental material includes gut content, fecal and cecal excrements, litter samples, as well as mixtures, solutions or suspensions thereof. An example for muscle tissue is breast (pectoralis major), examples for gut tissue are ileum and jejunum; and examples for organ tissue are spleen tissue or heart tissue. The term “litter sample” refers to mixed fecal droppings comprising residues of bedding material.

The biological sample obtained from the subject or from the population to be tested is preferably feces. Fecal sample material can be collected ante mortem. The DNA material isolated from feces contains significant amounts of gut cell DNA (mucosa).

In particular, the biological sample obtained from the subject or from the population to be tested is pooled fecal sample material deriving from a Galliformes population. Pooled fecal sample material is obtained by combining and mixing individual fecal samples.

The sample size (i.e. the number of excremental samples to be taken; each sample taken at a specific site within the animal house) has to be determined in view of the actual stocking density, i.e. with the actual number of animals belonging to the population to be tested.

In general, a minimum of 80 to 100 individual excremental samples are sufficient for most livestock chicken populations. As an example, for a broiler flock of 20000 animals, 96 individual samples are required for a confidence level of 95%.

For obtaining the pooled excremental sample material, several sampling methods may be used. In one example, the pooled excremental sample is obtained by systematic grid sampling (systematic random sampling). For this method, the animal house or area in which the avian population is kept is divided in a grid pattern of uniform cells or sub-areas based on the desired number of individual excremental samples (i.e., the sample size). Then, a random sample collection site is identified within the first grid cell and a first sample is taken at said site. Finally, further samples are obtained from adjacent cells sequentially—e.g., in a serpentine, angular or zig-zag fashion-using the same relative location within each cell. A random starting point can be obtained with a dice or a random number generator. The above process may optionally be repeated for replicate samples.

The genomic DNA used according to any aspect of the present invention is extracted or contained in the plurality of different sample materials deriving from various subjects of the Galliformes family and representing specific time points within the chronological lifespan of the specific species of the Galliformes family. As an example, the sample material may be stratified into four tissue (breast, ileum, spleen and jejunum) and three age (3 d, 15 d, 34 d) groups. Ideally, the sample material covers the entire life cycle of the species under investigation.

In particular, the plurality of different biological sample materials deriving from the subjects of the Galliformes family and representing specific time points within the chronological lifespan of this species may include material selected from the group consisting of body fluids, excremental material, tissue material and feather material. In one example, the plurality of different biological sample materials deriving from the subjects of the Galliformes family and representing specific time points within the chronological lifespan of this species/family includes only one specific tissue, or maximally four different tissues. In another example, the plurality of different biological sample materials deriving from the specific species and representing specific time points within the chronological lifespan of this species includes at least four different tissues and particularly exactly four different tissues.

In another example, the plurality of different biological sample materials deriving from the subjects of the Galliformes family and representing specific time points within the chronological lifespan of this avian species comprises or consists of tissue material selected from muscle tissue; organ tissue, such as gut tissue; and skin tissue.

In particular, the plurality of different biological sample materials deriving from the avian species and representing specific time points within the chronological lifespan of this avian species includes or consists of breast tissue, spleen tissue, ileum tissue and jejunum tissue. This set of tissues may be used as it represents a biologically diverse and commercially relevant set of tissues.

The plurality of different biological sample materials deriving from any species of the Galliformes family and representing specific time points within the chronological lifespan of avian species is particularly selected to represent ages ranging between day 3 and day 63, in particular between day 4 and day 42 and more in particular between day 5 and day 35. For example, the life cycle of chicken starts with eggs taken from parent birds in the hatchery which are then incubated at a constant temperature for 21 days until the bird hatches, though at this stage the precocial chicken might be up to 72 hours old they are called one-day chicken. These chickens are separated by sexes and the female birds are kept for approx. one year for laying eggs. The lifespan for broiler chicken is significantly shorter and varies between 21 days and up to 170 days. An average US broiler is slaughtered after 47 days at a slaughter weight of 2.6 kg while in Europe the average slaughter age is at 42 days (at a weight of 2.5 kg).

In the method according to any aspect of the present invention, in step (c), all CpG sites associated with single nucleotide polymorphisms (SNPs) were excluded from the CpG sites SNPs can be determined using standard procedures known in the art, such as whole-genome sequencing.

Alternatively, SNPs in the genome of selected species are publicly available in databases, such as dbSNP (https://www.ncbi.nlm.nih.gov/snp/).

‘Bisulfite treatment’ of genomic DNA, refers to the treatment of the genomic DNA with a deaminating agent such as a bisulfite that may be used to treat all DNA, methylated or not. In particular, the term “bisulfite” as used herein encompasses any suitable type of bisulfite, such as sodium bisulfite, or other chemical agents that are capable of chemically converting a cytosine (C) to an uracil (U) without chemically modifying a methylated cytosine and therefore can be used to differentially modify a DNA sequence based on the methylation status of the DNA, e.g., U.S. Pat. Pub. US 2010/0112595. As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

Accordingly, in step (a) according to any aspect of the present invention, the genomic DNA contained/obtained or extracted from the sample, is first bisulfite treated.

An alternative method available in the art may be used instead of bisulfite treatment. A skilled person will understand which other methods to use. In one example, TET-assisted pyridine borane sequencing (TAPS) may be used for detection of 5mC and 5hmC (Yibin Liu, et al., Nature Biotechnology, 37:424-429 (2019).

The bisulfite treated genomic DNA from step (a) is then amplified with specific primers that target at least two Low Methylated Regions (LMR) in the genomic DNA from (a) to obtain at least one Polymerase chain reaction (PCR) product in step (b).

PCR is used to amplify at least 2 LMRs in the bisulfite treated genomic DNA. In particular, at least three, four, five or six LMRs are amplified using PCR in the bisulfite treated genomic DNA. The accuracy/precision of the results (i.e. determining the biological age of the subject) is higher when more LMRs are amplified in step (b). That is to say amplifying six LMRs in step (b) will result in determining a biological age that is closer to reality than amplifying say 5, 4, 3, or 2 LMRs in step (b). However, with two LMRs amplified in step (b), the results are good enough to accurately predict the biological age of the subject.

During the PCR step in (b), usually more than one pair of primers are used for each LMR. Each pair of primers target a different region of the LMR thus resulting in more than one PCR product/amplicon for each LMR. In particular, each PCR product has a size in the range of 150 bp to 250 bp. In particular, the size of the PCR product is about 200 bp. Any method known in the art may be used to construct the sequences of the primers. In particular, the primers are specific to targeting specific regions within the LMRs in the bisulfite treated genomic DNA from step (a). Primer design using bisulfite converted DNA as template is difficult since it contains repeated Ts in the DNA sequences which might lead to non-specific binding of the primer sets. Since the amplicon size is so small and the DNA to be amplified first goes through bisulfite treatment before PCR is carried out, there are many non-specific products produced post PCR and there are many repetitive sequences produced in the DNA to be amplified by PCR respectively. This means to ensure that the PCR is carried out efficiently and effectively, optimization of the primers has to be carried out meticulously. A skilled person would be able to easily vary the conditions in the PCR to obtain suitable conditions to amplify the target regions of the LMRs to obtain the PCR product of the step (b). In one example, the PCR annealing temperature in 50° C.

Low Methylated Region (LMR) is a region of the genome wherein less than 60% of CpGs in that region are methylated. More in particular, less than 50%, 40%, 30%, 20% or 10% of the CpGs in the LMRs are methylated. Any method known in the art may be used to identify or detect LMRs in the genomic DNA. Well known methods include using programmes such as MethylSeekR. In particular, LMRs in the genomic DNA have at least three consecutive CpGs and have no single nucleotide polymorphisms (SNPs) in any of the CpG positions. Even more in particular, LMRs in the genomic DNA are identified based on the method disclosed at least in Burger, L., (2013) Nucleic Acids Research, 41 (16): e155 and/or Stadler, M., (2011) Nature 480, 490-495. LMRs are known to have an average methylation ranging from 10% to 50%; are regions of low CG density; tend to be enriched for H3K4me1, DHSs, and p300/CBP; and/or are primarily located distal to promoters in intergenic or intronic regions. In particular, LMRs:
have an average methylation ranging from 10% to 50%,
are regions of low CG density;
are enriched for Histone H3 monomethylated at lysine 4 (H3K4me1), DNase I hypersensitive sites (DHSs) and transcriptional coactivators CREB binding protein (CPB) and p300;
are primarily located distal to promoters in intergenic or intronic regions; and/or
have no single nucleotide polymorphisms (SNPs) in any of the CpG positions.

The specific CpG sites used in step (c) according to any aspect of the present invention is within the genomic DNA of any species of Galliformes family and are distributed within the LMRs in the genome of the species. In this case, method according to any aspect of the present invention, also includes the step of computing LMRs individually for the different tissues. MethylSeekR may be used to carry out this function as well. For establishing an LMR based mini clock, the specific CpG sites within the genomic DNA of any species of Galliformes family were restricted to a strand-specific coverage of at least greater than 5. An LMR based mini clock allows the conceptual interpretation of the selected features, as LMRs represent transcription factor binding sites. This represents an important advantage compared to all-CpG clocks. Furthermore, LMR based mini clocks are more robust to noise, as the features represent averages over regions and noise cancels out. Also, a mini clock provides a minimum list of LMRs that can be used to accurately determine the epigenetic age of the sample.

In addition to the above, the present invention pertains to a computer program loaded into a memory of a computer, implementing any one of the above-described method.

These LMRs identified in the bisulfite treated genomic DNA from step (a) are then amplified with specific primers using PCR.

In particular, the LMR in step (b) according to any aspect of the present invention is selected from a list of the following six LMRs to obtain a PCR product:

| LMR | chrom | start | end |
|---|---|---|---|
| 1 | chr12 | 9433041 | 9433568 |
| 2 | chr13 | 13146981 | 13147888 |
| 3 | chr20 | 11718628 | 11718916 |
| 4 | chr2 | 31316251 | 31316368 |
| 5 | chr2 | 91174538 | 91175128 |
| 6 | chr6 | 8416237 | 8416588 |

In one example, the two LMRs according to any aspect of the present invention may be LMR 1 corresponding to about base pair 9433041 to about base pair 9433568 of chicken chromosome 12 and LMR 5 corresponding to about base pair 91174538 to about base pair 91175128 chicken chromosome 2.

The two LMRs may be:
LMR 1 corresponding to about base pair 9433041 to about base pair 9433568 of chicken chromosome 12 and LMR 5 corresponding to about base pair 91174538 to about base pair 91175128 of chicken chromosome 2; or
LMR 1 corresponding to about base pair 9433041 to about base pair 9433568 of chicken chromosome 12 and LMR 3 corresponding to about base pair 11718628 to about base pair 11718916 of chicken chromosome 20, or
LMR 3 corresponding to about base pair 11718628 to about base pair 11718916 of chicken chromosome 20 and LMR 5 corresponding to about base pair 91174538 to about base pair 91175128 of chicken chromosome 2, or
LMR 2 corresponding to about base pair 13146981 to about base pair 13147888 of chicken chromosome 13 and LMR 6 corresponding to about base pair 8416237 to about base pair 8416588 of chicken chromosome 6.

In another example, three LMRs may be amplified in step (b) according to any aspect of the present invention and the three LMRs may be LMR 1 corresponding to about base pair 9433041 to about base pair 9433568 of chicken chromosome 12, LMR 3 corresponding to about base pair 11718628 to about base pair 11718916 of chicken chromosome 20 and LMR 5 corresponding to about base pair 91174538 to about base pair 91175128 of chicken chromosome 2.

The three LMRs may also be:
LMR 1, LMR 2 and LMR 5, or
LMR 1, LMR 3 and LMR 4, or
LMR 1, LMR 4, and LMR 5, or
LMR 1, LMR 5 and LMR 6.

In yet another example, four LMRs may be amplified in step (b) according to any aspect of the present invention and the four LMRs may be LMR 1 corresponding to about base pair 9433041 to about base pair 9433568 of chicken chromosome 12, LMR 3 corresponding to about base pair 11718628 to about base pair 11718916 of chicken chromosome 20, LMR 4 corresponding to about base pair 31316251 to about base pair 31316368 of chicken chromosome 2 and LMR 5 corresponding to about base pair 91174538 to about base pair 91175128 of chicken chromosome 2.

The four LMRs may also be:
LMR 1, LMR 2, LMR 3 and LMR 4, or
LMR 1, LMR 2, LMR 3 and LMR 5, or
LMR 1, LMR 2, LMR 3 and LMR 6, or
LMR 1, LMR 2, LMR 4 and LMR 5, or
LMR 1, LMR 2, LMR 4 and LMR 6, or
LMR 1, LMR 3, LMR 4 and LMR 5, or
LMR 1, LMR 3, LMR 4 and LMR 6, or
LMR 1, LMR 4, LMR 5 and LMR 6, or
LMR 2, LMR 3, LMR 4 and LMR 5, or
LMR 2, LMR 3, LMR 4 and LMR 6, or
LMR 3, LMR 4, LMR 5 and LMR 6.

In a further example, five LMRs may be amplified in step (b) according to any aspect of the present invention and the five LMRs may be LMR 1 corresponding to about base pair 9433041 to about base pair 9433568 of chicken chromosome 12, LMR 2 corresponding to about base pair 13146981 to about base pair 13147888 of chicken chromosome 13, LMR 3 corresponding to about base pair 11718628 to about base pair 11718916 of chicken chromosome 20, LMR 4 corresponding to about base pair 31316251 to about base pair 31316368 of chicken chromosome 2 and LMR 5 corresponding to about base pair 91174538 to about base pair 91175128 of chicken chromosome 2.

The five LMRs may also be:
LMR 1, LMR 2, LMR 3, LMR 4, and LMR 6 or
LMR 2, LMR 3, LMR 4, LMR 5, and LMR 6.

In yet another example, six LMRs may be amplified in step (b) according to any aspect of the present invention and the six LMRs may be LMR 1, LMR 2, LMR 3, LMR 4, LMR 5 and LMR 6. In step (c) according to any aspect of the present invention, the methylation level of CpG sites in the PCR product of step (b) is determined.

The term “CpG site”, “clock CpG” or “CpG location” as used in the context of the present invention refers to a CpG position that is potentially methylated, in particular, a nucleotide or sequence of nucleotides within a nucleic acid that is susceptible to methylation either by natural occurring events in vivo or by an event instituted to chemically methylate the nucleotide in vitro. Methylation typically occurs in a CpG containing nucleic acid. The CpG containing nucleic acid may be present in, e.g. a CpG island, a CpG doublet, a promoter, an intron, or an exon of a gene or in an intergenic region. For instance, the potential methylation sites may encompass the promoter/enhancer regions of the indicated genes.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated.

As used herein, a “methylated nucleic acid molecule” refers to a nucleic acid molecule that contains one or more nucleotides that is/are methylated.

Methylation levels of a set of the specific CpG sites can be normalized tissue-specifically. Normalization is performed by computing for every CpG the average methylation value over all samples from the same tissue and subtracting the thus-obtained value from the value of this CpG (or, alternatively by computing for every LMR the average methylation value over all samples from the same tissue and subtracting the thus-obtained value from the value of this LMR). This normalization is necessitated by the different aging trajectories of individual tissues.

That is, measuring DNA methylation at the thus-obtained CpG sites enables determining or establishing the epigenetic age of chicken and making accurate predictions of the chronological age of a subject of the Galliformes family according to any aspect of the present invention.

The term “methylation level” refers to the level of a specific methylation site which can range from 0 (=unmethylated) to 1 (=fully methylated). Thus, based on the methylation level of one or more methylation sites, a methylation profile may be determined. Accordingly, the term “methylation” profile” or also “methylation pattern” refers to the relative or absolute concentration of methylated C or unmethylated C at any particular stretch of residues in a biological sample. For example, if cytosine (C) residue(s) not typically methylated within a DNA sequence are more methylated in a sample, it may be referred to as “hypermethylated”; whereas if cytosine (C) residue(s) typically methylated within a DNA sequence are less methylated, it may be referred to as “hypomethylated”. Likewise, if the cytosine (C) residue(s) within a DNA sequence (e.g., sample nucleic acid) are more methylated when compared to another sequence from a different region or from a different individual (e.g., relative to normal nucleic acid), that sequence is considered hypermethylated compared to the other sequence. Alternatively, if the cytosine (C) residue(s) within a DNA sequence are less methylated as compared to another sequence from a different region or from a different individual, that sequence is considered hypomethylated compared to the other sequence. These sequences are said to be “differentially methylated”. For example, when the methylation status differs between inflamed and non-inflamed tissues, the sequences are considered “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

Whole genome bisulfite sequencing is a genome-wide analysis of DNA methylation based on the sodium bisulfite conversion of genomic DNA, which is then sequenced on a next-generation sequencing platform. The sequences are then aligned to the reference genome to determine methylation states of the CpG dinucleotides based on mismatches resulting from the conversion of unmethylated cytosines into uracil. For example, methylation levels can be measured using commercial Illumina™ sequencing or array platforms.

To quantify the methylation level, various established protocols may be used to calculate the beta value of methylation, which equals the fraction of methylated cytosines in a specific location. Step (c) may be performed with a mathematical algorithm and in particular with a statistical prediction method.

The selection of the CpGs, which define the mini clock, i.e. the set of specific CpG sites of step (c), may be done with a penalized regression. In this case, the evaluation of a newly sequenced test sample is done by evaluating the methylation values applying the existing regression function of the LMR based mini clock. In accordance therewith, a trained regression function is preferably applied in step (c).

In particular, the Galliformes subject or population to be tested is/are broiler(s) having a life span of up to 63 days.

In particular, the methylation level in step (c) is determined using a method selected from the group consisting of DNA pyrosequencing, mass spectrometry based (Epityper™), PCR based methylation assays, targeted-amplicon next generation bisulfite sequencing on a platform selected from a group of HiSeq, MiniSeq, MiSeq, and NextSeq sequencers, Ion Torrent sequencing, methylated DNA Immunoprecipitation (MeDIP) sequencing, and hybridization with oligonucleotide arrays.

More in particular, the average methylation value of each of the LMRs of the test DNA sample based on methyl ratios of the CpG sites is calculated. In one example, the amplification of the LMR from a bisulfite treated biological sample in step (b), leads to the production of more than one PCR product. The methylation value of the CpG sites in all the PCR products from the single LMR is measured in step (c). This value together with the weight of the whole LMR is used in a computation to determine the epigenetic age of the sample in step (d) according to any aspect of the present invention. In particular, the biological age is computed by summation of the products of average methylation value and weight of the LMRs. More in particular, in step (d) biological age of the subject is determined with a statistical prediction algorithm, wherein the statistical prediction algorithm is applied to the measured methylation level to determine the epigenetic age of the subject. In step (d), the statistical prediction algorithm comprises
(i) obtaining a linear combination of the measured methylation level of the CpG sites in the PCR product, and
(ii) applying a transformation to the linear combination to determine the biological age of the subject.

A more detailed description of this step in disclosed in WO2015048665A2.

Once the epigenetic age of the test subject is determined, comparing the epigenetic age with the chronological age may allow one to presume the welfare conditions of the test subject.

The epigenetic age generally depends on the biological state or condition of an individual (or of a population). Epigenetic age may match or mismatch with chronological age. Deviations of the epigenetic age from the chronological age are age acceleration or age deceleration. Accordingly, epigenetic age may also be determined by comparison of the methylation levels of the methylation markers (i.e. CpG sites) in the genomic Galliformes DNA from the sample to be tested with the methylation status of the same markers (i.e. CpG sites) from an age-correlated reference sample in the LMRs.

In particular, the method according to any aspect of the present invention may be relevant for any subject of the Galliformes family. More in particular, the method is relevant for turkey, chicken, quail, and pheasant. Even more in particular, the method according to any aspect of the present invention is relevant for chicken (Gallus gallus domesticus).

In one example, in chicken (Gallus gallus domesticus), a mismatch of epigenetic and chronological age, and in particular epigenetic age acceleration (i.e. epigenetic age>chronological age) is an early indication of inflammatory processes.

Accordingly, based on the methods according to any aspect of the present invention, necessity of therapeutic or nutritional interventions may be evaluated based thereon. Such intervention may include providing an individualized (tailored) treatment to the individual or population tested to bring the predicted epigenetic age closer to the chronological age of the individual or population.

Such intervention may include providing an individualized (tailored) treatment to the individual or population tested to bring the predicted epigenetic age closer to the chronological age of the individual or population. Further, such treatment or intervention may include feeding or administering health-promoting substances, such as zootechnical feed additives, or therapeutic agents. The term “administering” or related terms includes oral administration. Oral administration may be via drinking water, oral gavage, aerosol spray or animal feed. The term “zootechnical feed additive” refers to any additive used to affect favorably the performance of animals in good health or used to affect favorably the environment. Examples for zootechnical feed additives are digestibility enhancers, i.e. substances which, when fed to animals, increase the digestibility of the diet, through action on target feed materials; gut flora stabilizers; microorganisms or other chemically defined substances, which, when fed to animals, have a positive effect on the gut flora; or substances which favorably affect the environment. In particular, the health-promoting substances are selected from the group consisting of probiotic agents, prebiotic agents, botanicals, organic/fatty acids, bacteriophages and bacteriolytic enzymes or any combinations thereof. In addition to the above, the present invention also pertains to the use of the methods disclosed herein for the development of a routine analysis tool such as real-time PCR, targeted sequencing/panel sequencing, methylated DNA immunoprecipitation as input for both, chip/array technology or methylated DNA sequencing.

Applications of the methods according to the invention are for example ((i) aiding in evaluation of the health status of Galliformes, such as chicken (ii) monitoring the progress or reoccurrence of clinical and sub-clinical disorders or (iii) studying the effects of medication, feed compounds and/or special diets on the biological age—and thus on the health status of Galliformes, such as chicken.

Applications of the methods according to any aspect of the present invention in particular help to avoid loss in animal performance like weight gain and feed conversion.

According to another aspect of the present invention, there is provided An in vitro method for estimating the inflammation status in Galliformes, the method comprising the steps of:
(a) bisulfite treatment of genomic DNA extracted from a biological sample obtained from the subject;
(b) amplifying the bisulfite treated genomic DNA from step (a) with specific primers that target at least two Low Methylated Region (LMR) to obtain a PCR product;
(c) measuring the methylation level of CpG sites in the PCR product of step (b);
(d) determining the biological age of the subject with a statistical prediction algorithm, wherein the statistical prediction algorithm is applied to the measured methylation level to determine the epigenetic age of the subject; and
   wherein the subject is from the Galliformes family and wherein an epigenetic age higher than the chronological age is indicative of inflammation,
   with a proviso that the CpG sites associated with single nucleotide polymorphisms are not considered.

In particular, the subject is from the Gallus gallus domesticus species.

The LMR in step (b) according to any aspect of the present invention is selected from a list of the following six LMRs to obtain a PCR product:

| LMR | chrom | start | end |
|---|---|---|---|
| 1 | chr12 | 9433041 | 9433568 |
| 2 | chr13 | 13146981 | 13147888 |
| 3 | chr20 | 11718628 | 11718916 |
| 4 | chr2 | 31316251 | 31316368 |
| 5 | chr2 | 91174538 | 91175128 |
| 6 | chr6 | 8416237 | 8416588 |

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Determining Epigenetic Age of Samples of Gallus gallus Domesticus Species Using PCR
Strategy for Construction and Evaluation of Mini-Clocks (with Low Number of LMRs)

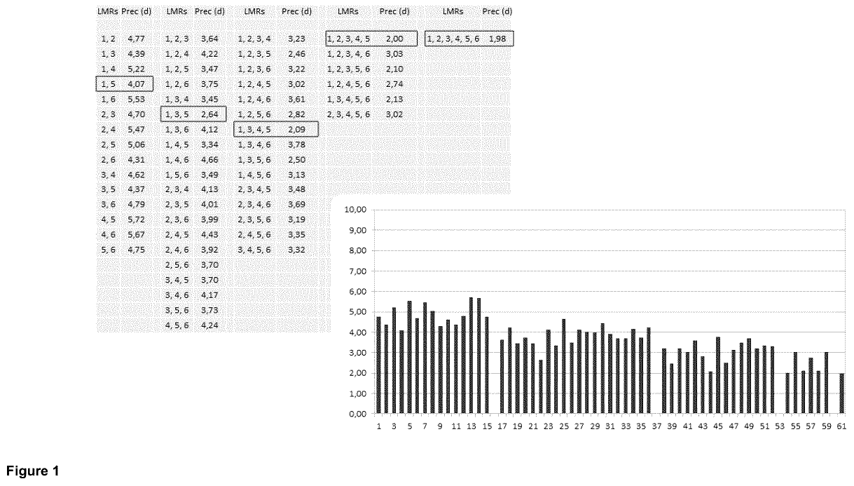

6 LMRs with highest absolute weights as starting set from the LMR clock with 32 LMRs (Raddatz, G., et al, Commun Biol 4, 76 (2021)) were selected. All possible subsets of 2, 3, 4, 5 or 6 LMRs from this set of 6, were selected resulting in 57 different sets of LMRs set with alpha=0.9. Then, a DNA mini clock for each of 57 sets of LMRs was constructed resulting in 57 clocks altogether by normalization and training similar to the 32-LMR clock (Raddatz, G., et al, Commun Biol 4, 76 (2021)). The precision of for each of the 57 clocks was then evaluated and results shown in FIG. 1.

In particular, the absolute weights of the 6 LMRs are provided below.

| LMR | chrom | start | end | weight |
|---|---|---|---|---|
| 1 | chr12 | 9433041 | 9433568 | 9,905 |
| 2 | chr13 | 13146981 | 13147888 | −10,892 |
| 3 | chr20 | 11718628 | 11718916 | 20,167 |
| 4 | chr2 | 31316251 | 31316368 | 15,824 |
| 5 | chr2 | 91174538 | 91175128 | −26,554 |
| 6 | chr6 | 8416237 | 8416588 | 12,93 |

### PCR Analysis of Test Samples of Chicken

Chicken tissue samples are collected from a customer. Genomic DNA is purified from the tissue samples using the DNeasy Blood & Tissue Kit (Qiagen) and is quantified using the PicroGreen or NanoDrop™ 2000.

The genomic DNA (500 ng) from tissue samples are subjected to bisulfite conversion using the EZ DNA Methylation-Gold™ Kit (Zymo Research).

Specific primer sets (Forward primer and Reverse primer) were designed for PCR amplification of different regions of the LMRs using bisulfite-converted genomic DNA as a template. The primers were 26 to 32 bases long and did not contain any CpG sites. A universal Ftag (SEQ ID NO:37) and Rtag (SEQ ID NO:38) sequences were added at the 5′ end of the forward and reverse primers, respectively. CpGs that are found to be SNPs via comparative analysis in published sequences were not being considered for analysis. The list of primers used is provided in Table 1 below.

For the primer design, all Cs were in the template DNA treated as Ts. The amplicon sizes were relatively short (between 150-250 bp), hence, 2-6 pairs of primers were designed for each LMR to cover all the relevant CpG sites. The primer design was optimized in such a way that the primer set specifically binds to the desired region of a particular LMR.

The PCR is carried out using PyroMark PCR Kit (Qiagen) under suitable conditions where templates are most efficiently amplified to avoid high cycle numbers that may lead to a bias. For primary PCR, around 20 ng of bisulfite converted genomic DNA was used as a template in 25 μl PCR reactions. The PCR condition is listed below.

| | |
|---|---|
| PCR parameters | PyroMark PCR Master Mix |

| | | | |
|---|---|---|---|
| Initial denaturation | 95° C. for 15 min | —— | |
| Denaturation | 94° C. for 30 seconds | | |
| Annealing | 56° C. for 30 seconds | | ×45 |
| Extension | 72° C. for 30 seconds | | |
| Final extension | 72° C. for 10 min | | |
| Hold | 4° C. | | |

The PCR samples are then analyzed by electrophoresis on 2% agarose and PCR amplified products were compared with DNA standard markers. The PCR-amplified products are initially analyzed by Sanger sequencing to verify the specificity of the primer sets to respective LMRs.

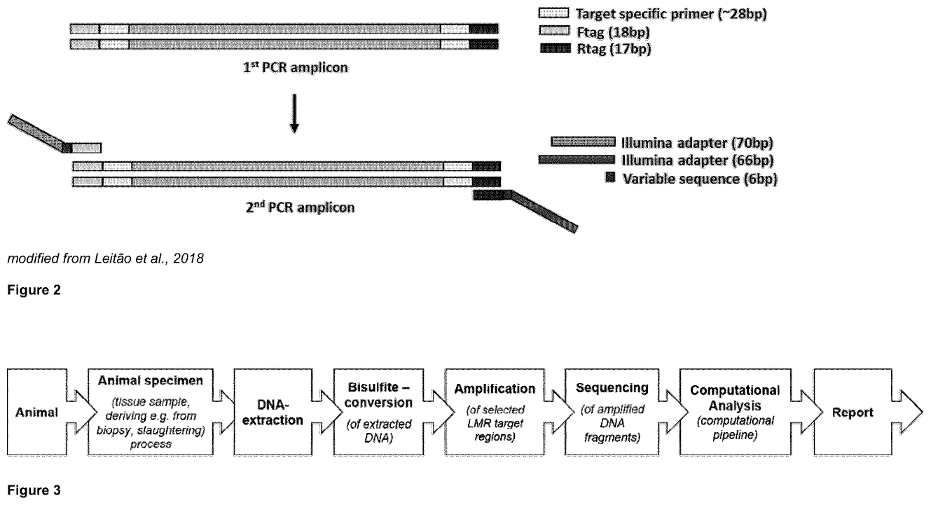

To quantitatively analyze methylation of each CpGs within the LMRs, a second round of PCR was performed using 2 μl of the first-round PCR product and amplified using the PyroMark PCR Kit (Qiagen). PCR primers for secondary PCRs include Ftag/Rtag, Illumina adapters and a variable stretch of 6 bp as shown in FIG. 2 (Leitão et al., Methods Mol Biol, 1767:351-366 (2018)).

The PCR products from the second PCR corresponding to 6 LMRs were pooled together and purified using Agencourt AMPure beads XP according to the manufacturer's protocol. The purified PCR products are then analyzed by a quantitative NGS using NovaSeq platform.

TABLE 1
Primers used in the PCR step to amplify LMRs- LMR 1- LMR 6

| | Genomic | No | | | Product | No of | |
|---|---|---|---|---|---|---|---|
| | region | of | | | Size | CpGs | SEQ ID |
| LMR | (bp) | CpGs | Primer Name | Sequence | (bp) | covered | NO: |
| 1 | 527 | 10 | Chr12_Fwd1 | GAAAAGTAGAAAGTATTTTAATTATTG | 172 | 4 | 1 |
| | | | Chr12_Rev1 | CCCTTAAAAATAACTCATATTTACTTA | | | 2 |
| | | | Chr12_Fwd2 | GTAGTAATTTAAGGTAGTTTTTAGTTTG | 137 | 4 | 3 |
| | | | Chr12_Rev2 | CTATTACACATTACTTTTATAAAAAAC | | | 4 |
| | | | Chr12_Fwd3 | GTTTTTTATAAAAGTAATGTGTAATAG | 108 | 2 | 5 |
| | | | Chr12_Rev3 | CCAATCTTTTATTAAATTTAACTAATC | | | 6 |
| 2 | 907 | 40 | Chr13_Fwd1 | GGTAGTAGTGGGGTGTTTTTTATTG | 218 | 8 | 7 |
| | | | Chr13_Rev1 | CAACCAACCCACAACTACTCTCTAAC | | | 8 |
| | | | Chr13_Fwd2 | GTTAGAGAGTAGTTGTGGGTTGGTTG | 178 | 6 | 9 |
| | | | Chr13_Rev2 | CAACCAAACTTACAATCTATCAAATAAAC | | | 10 |
| | | | Chr13_Fwd3 | GTTGGTATAGTGGGGTATTTTTATG | 146 | 2 | 11 |
| | | | Chr13_Rev3 | CCAAAAACTAATAATATCAATACTCC | | | 12 |
| | | | Chr13_Fwd4 | GGAGTATTGATATTATTAGTTTTTGG | 137 | 4 | 13 |
| | | | Chr13_Rev4 | TAACTCTAAACTCTATCCTACCCCC | | | 14 |
| | | | Chr13_Fwd5 | GGGGTAGGATAGAGTTTAGAGTTA | 167 | 10 | 15 |
| | | | Chr13_Rev5 | CAATACTTAAATAACTACCACACAC | | | 16 |
| | | | Chr13_Fwd6 | GTGTGTGGTAGTTATTTAAGTATTG | 166 | 10 | 17 |
| | | | Chr13_Rev6 | CACCAAACACCTTACAACTACC | | | 18 |
| 3 | 288 | 20 | Chr20_Fwd1 | GTTAGTTTTTTTGTTGAGTATATTTCG | 194 | 10 | 19 |
| | | | Chr20_Rev1 | CAACTCACTAATTTCAAATCAC | | | 20 |
| | | | Chr20_Fwd2 | GTGATTTGAAATTAGTGAGTTG | 125 | 10 | 21 |
| | | | Chr20_Rev2 | CACCCAAAAATACTAAAATAAACAAC | | | 22 |
| 4 | 117 | 8 | LMR4_Fwd1 | GTTTTTAAAATGAGGGTTGTAATGG | 153 | 6 | 23 |
| | | | LMR4_Rev1 | CAAAAAACCTAATATACACCAAATAATAAC | | | 24 |
| | | | LMR4_Fwd2 | GTTATTATTTGGTGTATATTAGGTTTTTTG | 136 | 2 | 25 |
| | | | LMR4_Rev2 | CCAAAATAACAATTCAATAAACCTC | | | 26 |
| 5 | 590 | 6 | Chr2_Fwd1 | GGATAGAGGAGGAAATAAGTTTTATTG | 111 | 2 | 27 |
| | | | Chr2_Rev1 | CTATCAAATACAACAAAATCTATCCTATC | | | 28 |
| | | | Chr2_Fwd2 | GATTTAGAAAGTTATTTGAGAAATTTAAG | 185 | 4 | 29 |
| | | | Chr2_Rev2 | CACCTCCTATAAATAACAATACAATC | | | 30 |
| 6 | 351 | 50 | Chr6_Fwd1 | GTTATTGAAAGTAGGGTAGTGATGGATG | 165 | 12 | 31 |
| | | | Chr6_Rev1 | CACCAAATACAATACCTCTAAAACC | | | 32 |
| | | | Chr6_Fwd2 | GGTTTTAGAGGTATTGTATTTGGTG | 156 | 24 | 33 |
| | | | Chr6_Rev2 | CAACCCTACAACAAACAAATTAAAAAC | | | 34 |
| | | | Chr6_Fwd3 | GTTTTTAATTTGTTTGTTGTAGGGTTG | 139 | 14 | 35 |
| | | | Chr6_Rev3 | TTTCAAACTCTACAAAATCCTACCTC | | | 36 |

Biological Age Evaluation with Mini Clock

### Processing:

Sequenced reads were trimmed and mapped with BSMAP version 2.5 using the assembly version 5.0 of the chicken (Gallus gallus) genome as reference sequence. Methylation ratios were determined using a Python script (methratio.py) distributed with the BSMAP package. For all further analysis, only CpGs covered by at least five reads were considered.

### Age Estimation

The average methylation value of the each of the LMRs in the DNA mini clock based on methyl ratios of the CpG sites is calculated. The biological age is computed by summation of the products of average methylation value and weight of the LMRs using the formula provided below.

The biological age of the chicken is then calculated with the following formula

$$Biological age = {\sum }_{i = 1}^{n} {a}_{i} {w}_{i}$$

where, i is the LMR, a is the average methylation of the LMR; w is the weight computed for the LMR (this weight of the LMR is specific to this mini clock), n is 1-6.

The method to determine the weight of the LMR is provided in Raddatz, G., et al, Commun Biol 4, 76 (2021).

The biological age of the chicken is then compared to the chronological age of the chicken.

1. An in vitro method for predicting the biological age of a subject, the method comprising the steps of:
(a) bisulfite treatment of genomic DNA extracted from a biological sample obtained from the subject;
(b) amplifying the bisulfite treated genomic DNA from step (a) with specific primers that target at least two Low Methylated Region (LMR) in the genomic DNA from (a) to obtain at least one PCR product;
(c) measuring the methylation level of CpG sites in the PCR product of step (b);
(d) determining the biological age of the subject with a statistical prediction algorithm, wherein the statistical prediction algorithm is applied to the measured methylation level to determine the epigenetic age of the subject;
wherein the subject is from the Galliformes family and
wherein at least three LMRs are amplified in step (b),
with a proviso that the CpG sites associated with single nucleotide polymorphisms are not considered.

2. The method according to claim 1, wherein at least four LMRs are amplified in step (b).

3. The method according to claim 1, wherein in step (d), the statistical prediction algorithm comprises
(i) obtaining a linear combination of the measured methylation level of the CpG sites in the PCR product, and
(ii) applying a transformation to the linear combination to determine the biological age of the subject.

4. The method according to claim 1, wherein each of the forward and reverse primers of the specific primers used in step (b) are at least 26 to 32 bases long and do not contain any CpG sites.

5. The method according to claim 1, wherein the subject is from the Gallus gallus domesticus species.

6. The method according to claim 1, wherein the LMRs in step (b) is selected from a list of the following six LMRs to obtain a PCR product:

| LMR | chrom | start | end |
|---|---|---|---|
| 1 | chr12 | 9433041 | 9433568 |
| 2 | chr13 | 13146981 | 13147888 |
| 3 | chr20 | 11718628 | 11718916 |
| 4 | chr2 | 31316251 | 31316368 |
| 5 | chr2 | 91174538 | 91175128 |
| 6 | chr6 | 8416237 | 8416588. |

7. The method according to claim 6, wherein the two LMRs are LMR 1 corresponding to about base pair 9433041 to about base pair 9433568 of chicken chromosome 12 and LMR 5 corresponding to about base pair 91174538 to about base pair −26,554 of chicken chromosome 2.

8. The method according to claim 6, wherein the 3 LMRs are LMR 1 corresponding to about base pair 9433041 to about base pair 9433568 of chicken chromosome 12, LMR 5 corresponding to about base pair 91174538 to about base pair −26,554 of chicken chromosome 2 and LMR 3 corresponding to about base pair 11718628 to about base pair 11718916 of chicken chromosome 20.

9. The method according to claim 6, wherein the 4 LMRs are LMR 1 corresponding to about base pair 9433041 to about base pair 9433568 of chicken chromosome 12, LMR 5 corresponding to about base pair 91174538 to about base pair −26,554 of chicken chromosome 2, LMR 3 corresponding to about base pair 11718628 to about base pair 11718916 of chicken chromosome 20 and LMR 4 corresponding to about base pair 31316251 to about base pair 31316368 of chicken chromosome 2.

10. The method according to claim 1, wherein the biological sample obtained from the subject is selected from the group consisting of body fluids, excremental material, tissue material, feather material and combinations thereof.

11. An in vitro method for estimating the inflammation status in Galliformes, the method comprising the steps of:
(a) bisulfite treatment of genomic DNA extracted from a biological sample obtained from the subject;
(b) amplifying the bisulfite treated genomic DNA from step (a) with specific primers that target at least two Low Methylated Region (LMR) to obtain a PCR product;
(c) measuring the methylation level of CpG sites in the PCR product of step (b);
(d) determining the biological age of the subject with a statistical prediction algorithm, wherein the statistical prediction algorithm is applied to the measured methylation level to determine the epigenetic age of the subject; and
wherein the subject is from the Galliformes family and wherein an epigenetic age higher than the chronological age is indicative of inflammation, and
wherein at least three LMRs are amplified in step (b),
with a proviso that the CpG sites associated with single nucleotide polymorphisms are not considered.

12. The method according to claim 11, wherein the subject is from the Gallus gallus domesticus species.

13. The method according to claim 12, wherein the LMR in step (b) is selected from a list of the following six LMRs to obtain a PCR product:

| LMR | chrom | start | end |
|---|---|---|---|
| 1 | chr12 | 9433041 | 9433568 |
| 2 | chr13 | 13146981 | 13147888 |
| 3 | chr20 | 11718628 | 11718916 |
| 4 | chr2 | 31316251 | 31316368 |
| 5 | chr2 | 91174538 | 91175128 |
| 6 | chr6 | 8416237 | 8416588. |

14. The method according to claim 11, wherein each of the forward and reverse primers of the specific primers used in step (b) are at least 26 to 32 bases long and do not contain any CpG sites.


SEQUENCE LISTING (36 sequences)

SEQ ID NO: 1 - DNA, 27 bp, synthetic construct
  misc_feature 1..27: note = Chr12_Fwd1
  source 1..27: mol_type = other DNA
gaaaagtagaaagtattttaattattg

SEQ ID NO: 2 - DNA, 27 bp, synthetic construct
  misc_feature 1..27: note = Chr12_Rev1
  source 1..27: mol_type = other DNA
cccttaaaaataactcatatttactta

SEQ ID NO: 3 - DNA, 28 bp, synthetic construct
  misc_feature 1..28: note = Chr12_Fwd2
  source 1..28: mol_type = other DNA
gtagtaatttaaggtagtttttagtttg

SEQ ID NO: 4 - DNA, 27 bp, synthetic construct
  misc_feature 1..27: note = Chr12_Rev2
  source 1..27: mol_type = other DNA
ctattacacattacttttataaaaaac

SEQ ID NO: 5 - DNA, 27 bp, synthetic construct
  misc_feature 1..27: note = Chr12_Fwd3
  source 1..27: mol_type = other DNA
gttttttataaaagtaatgtgtaatag

SEQ ID NO: 6 - DNA, 27 bp, synthetic construct
  misc_feature 1..27: note = Chr12_Rev3
  source 1..27: mol_type = other DNA
ccaatcttttattaaatttaactaatc

SEQ ID NO: 7 - DNA, 25 bp, synthetic construct
  misc_feature 1..25: note = Chr13_Fwd1
  source 1..25: mol_type = other DNA
ggtagtagtggggtgttttttattg

SEQ ID NO: 8 - DNA, 26 bp, synthetic construct
  misc_feature 1..26: note = Chr13_Rev1
  source 1..26: mol_type = other DNA
caaccaacccacaactactctctaac

SEQ ID NO: 9 - DNA, 26 bp, synthetic construct
  misc_feature 1..26: note = Chr13_Fwd2
  source 1..26: mol_type = other DNA
gttagagagtagttgtgggttggttg

SEQ ID NO: 10 - DNA, 29 bp, synthetic construct
  misc_feature 1..29: note = Chr13_Rev2
  source 1..29: mol_type = other DNA
caaccaaacttacaatctatcaaataaac

SEQ ID NO: 11 - DNA, 25 bp, synthetic construct
  misc_feature 1..25: note = Chr13_Fwd3
  source 1..25: mol_type = other DNA
gttggtatagtggggtatttttatg

SEQ ID NO: 12 - DNA, 26 bp, synthetic construct
  misc_feature 1..26: note = Chr13_Rev3
  source 1..26: mol_type = other DNA
ccaaaaactaataatatcaatactcc

SEQ ID NO: 13 - DNA, 26 bp, synthetic construct
  misc_feature 1..26: note = Chr13_Fwd4
  source 1..26: mol_type = other DNA
ggagtattgatattattagtttttgg

SEQ ID NO: 14 - DNA, 25 bp, synthetic construct
  misc_feature 1..25: note = Chr13_Rev4
  source 1..25: mol_type = other DNA
taactctaaactctatcctaccccc

SEQ ID NO: 15 - DNA, 24 bp, synthetic construct
  misc_feature 1..24: note = Chr13_Fwd5
  source 1..24: mol_type = other DNA
ggggtaggatagagtttagagtta

SEQ ID NO: 16 - DNA, 25 bp, synthetic construct
  misc_feature 1..25: note = Chr13_Rev5
  source 1..25: mol_type = other DNA
caatacttaaataactaccacacac

SEQ ID NO: 17 - DNA, 25 bp, synthetic construct
  misc_feature 1..25: note = Chr13_Fwd6
  source 1..25: mol_type = other DNA
gtgtgtggtagttatttaagtattg

SEQ ID NO: 18 - DNA, 22 bp, synthetic construct
  misc_feature 1..22: note = Chr13_Rev6
  source 1..22: mol_type = other DNA
caccaaacaccttacaactacc

SEQ ID NO: 19 - DNA, 27 bp, synthetic construct
  misc_feature 1..27: note = Chr20_Fwd1
  source 1..27: mol_type = other DNA
gttagtttttttgttgagtatatttcg

SEQ ID NO: 20 - DNA, 22 bp, synthetic construct
  misc_feature 1..22: note = Chr20_Rev1
  source 1..22: mol_type = other DNA
caactcactaatttcaaatcac

SEQ ID NO: 21 - DNA, 22 bp, synthetic construct
  misc_feature 1..22: note = Chr20_Fwd2
  source 1..22: mol_type = other DNA
gtgatttgaaattagtgagttg

SEQ ID NO: 22 - DNA, 26 bp, synthetic construct
  misc_feature 1..26: note = Chr20_Rev2
  source 1..26: mol_type = other DNA
cacccaaaaatactaaaataaacaac

SEQ ID NO: 23 - DNA, 25 bp, synthetic construct
  misc_feature 1..25: note = LMR4_Fwd1
  source 1..25: mol_type = other DNA
gtttttaaaatgagggttgtaatgg

SEQ ID NO: 24 - DNA, 30 bp, synthetic construct
  misc_feature 1..30: note = LMR4_Rev1
  source 1..30: mol_type = other DNA
caaaaaacctaatatacaccaaataataac

SEQ ID NO: 25 - DNA, 30 bp, synthetic construct
  misc_feature 1..30: note = LMR4_Fwd2
  source 1..30: mol_type = other DNA
gttattatttggtgtatattaggttttttg

SEQ ID NO: 26 - DNA, 25 bp, synthetic construct
  misc_feature 1..25: note = LMR4_Rev2
  source 1..25: mol_type = other DNA
ccaaaataacaattcaataaacctc

SEQ ID NO: 27 - DNA, 27 bp, synthetic construct
  misc_feature 1..27: note = Chr2_Fwd1
  source 1..27: mol_type = other DNA
ggatagaggaggaaataagttttattg

SEQ ID NO: 28 - DNA, 29 bp, synthetic construct
  misc_feature 1..29: note = Chr2_Rev1
  source 1..29: mol_type = other DNA
ctatcaaatacaacaaaatctatcctatc

SEQ ID NO: 29 - DNA, 29 bp, synthetic construct
  misc_feature 1..29: note = Chr2_Fwd2
  source 1..29: mol_type = other DNA
gatttagaaagttatttgagaaatttaag

SEQ ID NO: 30 - DNA, 26 bp, synthetic construct
  misc_feature 1..26: note = Chr2_Rev2
  source 1..26: mol_type = other DNA
cacctcctataaataacaatacaatc

SEQ ID NO: 31 - DNA, 28 bp, synthetic construct
  misc_feature 1..28: note = Chr6_Fwd1
  source 1..28: mol_type = other DNA
gttattgaaagtagggtagtgatggatg

SEQ ID NO: 32 - DNA, 25 bp, synthetic construct
  misc_feature 1..25: note = Chr6_Rev1
  source 1..25: mol_type = other DNA
caccaaatacaatacctctaaaacc

SEQ ID NO: 33 - DNA, 25 bp, synthetic construct
  misc_feature 1..25: note = Chr6_Fwd2
  source 1..25: mol_type = other DNA
ggttttagaggtattgtatttggtg

SEQ ID NO: 34 - DNA, 27 bp, synthetic construct
  misc_feature 1..27: note = Chr6_Rev2
  source 1..27: mol_type = other DNA
caaccctacaacaaacaaattaaaaac

SEQ ID NO: 35 - DNA, 27 bp, synthetic construct
  misc_feature 1..27: note = Chr6_Fwd3
  source 1..27: mol_type = other DNA
gtttttaatttgtttgttgtagggttg

SEQ ID NO: 36 - DNA, 26 bp, synthetic construct
  misc_feature 1..26: note = Chr6_Rev3
  source 1..26: mol_type = other DNA
tttcaaactctacaaaatcctacctc